(19) United States
(12) Patent Application Publication
(10) Pub. No.: US 2020/0120312 A1
(43) Pub. Date: Apr. 16, 2020

(54) Systems And Methods For Capturing Images Based On Biorhythms

(71) Applicant: Plain Louie, LLC, St. Louis, MO (US)

(72) Inventors: Kristin Blume SLATER, St. Louis, MO (US); Rebecca Hamilton SCHULZ, St. Louis, MO (US)

(21) Appl. No.: 16/471,342

(22) PCT Filed: Dec. 19, 2017
(86) PCT No.: PCT/US2017/067396

§ 371 Date: Jun. 19, 2019

Related U.S. Application Data
(60) Provisional application No. 62/436,820, filed on Dec. 20, 2016.

(51) Int. Cl. H04N 7/18; A61B 5/00; A61B 5/024; A61B 5/053; A61B 5/021

(52) U.S. Cl. CPC H04N 7/188; H04N 7/185; H04N 7/181; A61B 5/0077; A61B 5/02427; A61B 5/6833; A61B 5/053; A61B 5/02141; A61B 5/681; A61B 5/6803; A61B 5/02405

(57) ABSTRACT

According to some aspects of the present disclosure, systems and methods for capturing images based on biorhythms are disclosed. An example system includes at least one processor configured to receive a biorhythm signal from a biosensor. The received biorhythm signal is indicative of at least one biorhythm of a user as detected by the biosensor. The system also includes a camera disposed remotely from the biosensor and user and adapted to capture one or more images when triggered by the at least one processor. The at least one processor is configured to compare the received biorhythm signal to a biorhythm threshold and, in response to the received biorhythm signal exceeding the biorhythm threshold, to trigger the camera to capture the one or more images.

### CROSS-REFERENCE TO RELATED APPLICATION

This application claims the benefit and priority of U.S. Provisional Application No. 62/436,820, filed on Dec. 20, 2016. The entire disclosure of the above application is incorporated herein by reference.

### FIELD

The present disclosure relates to systems and methods for capturing images based on biorhythms.

### BACKGROUND

This section provides background information related to the present disclosure which is not necessarily prior art.

Cameras are often used to capture images (e.g., pictures, videos, etc.). Typically, the user views a field of the camera lens through a viewfinder, a display screen, etc., and then presses a button to capture a desired image. This process requires manual operation and active control of the camera by the user. Separately, biosensors are sometimes used to monitor biorhythms of a user, such as heart rate.

### SUMMARY

This section provides a general summary of the disclosure, and is not a comprehensive disclosure of its full scope or all of its features.

According to one aspect of the present disclosure, a system for capturing images based on biorhythms includes at least one processor configured to receive a biorhythm signal from a biosensor. The received biorhythm signal is indicative of at least one biorhythm of a user as detected by the biosensor. The system also includes a camera disposed remotely from the biosensor and user and adapted to capture one or more images when triggered by the at least one processor. The at least one processor is configured to compare the received biorhythm signal to a biorhythm threshold and, in response to the received biorhythm signal exceeding the biorhythm threshold, to trigger the camera to capture the one or more images.

According to another aspect of the present disclosure, a system for capturing images based on biorhythms includes at least one processor configured to receive a biorhythm signal from a biosensor. The received biorhythm signal is indicative of at least one biorhythm of a user as detected by the biosensor. The system also includes a camera adapted to capture one or more images when triggered by the at least one processor, and a monitoring device configured to receive the one or more captured images. The at least one processor is configured to compare the received biorhythm signal to a biorhythm threshold, to trigger the camera to capture the one or more images in response to the received biorhythm signal exceeding the biorhythm threshold, and to transmit the one or more captured images to the monitoring device.

According to a further aspect of the present disclosure, a system for capturing images based on biorhythms includes at least one processor configured to receive a biorhythm signal from a biosensor. The received biorhythm signal is indicative of at least one biorhythm of a user as detected by the biosensor. The system also includes a computing device display adapted to display multiple screen images. The at least one processor is configured to compare the received biorhythm signal to a biorhythm threshold and, in response to the received biorhythm signal exceeding the biorhythm threshold, to capture a current screen image of the computing device display.

According to yet another aspect of the present disclosure, a method for capturing images based on biorhythms is disclosed. The method generally includes sensing a biorhythm of a user with a biosensor, and transmitting a biorhythm signal from the biosensor to at least one processor. The biorhythm signal is indicative of at least one biorhythm of a user as detected by the biosensor. The method also includes comparing the biorhythm signal to a biorhythm threshold, and, in response to the biorhythm signal exceeding the biorhythm threshold, the at least one processor performing at least one of: triggering a camera disposed remotely from the biosensor and the user to capture one or more images; triggering a wearable camera to capture one or more images and transmitting the one or more images captured by the wearable camera to the monitoring device; and capturing a current screen image of a computing device display.

Further aspects and areas of applicability will become apparent from the description provided herein. It should be understood that various aspects and features of this disclosure may be implemented individually or in combination with one or more other aspects or features. It should also be understood that the description and specific examples herein are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a diagram of a system for capturing images based on a user's biorhythms, according to one embodiment of the present disclosure.

FIG. 2 is a diagram of a system for capturing images based on a user's biorhythms using a remote camera, according to another embodiment of the present disclosure,

FIG. 3 is a block diagram of the system of FIG. 1, further including a sensor, a camera, a monitoring device and a server.

FIG. 4 is a block diagram of the system of FIG. 1, further including a sensor, a camera, a server, and two monitoring devices.

FIG. 5 is a block diagram of the system of FIG. 1, further including a sensor, a wearable camera, a stationary camera, and a server.

FIG. 6 is a block diagram of the system of FIG. 1, further including a sensor, a wearable camera, a stationary camera, a server and a monitoring device.

FIG. 7 is a block diagram of the system of FIG. 1, further including a sensor, a computer device display, and a server.

FIG. 8 is a flow chart of a method for capturing images based on a user's biorhythms, according to another embodiment of the present disclosure.

FIG. 9 is a flow chart of an example method for capturing images including a user wearing a wearable camera in an amusement park.

FIG. 10 is a flow chart of an example method for capturing images of students and teachers in a classroom using stationary cameras.

FIG. 11 is a flow chart of an example method for capturing images including a child wearing a wearable camera.

FIG. 12 is a flow chart of an example method of capturing images of a pet in the home.

FIG. 13 is a flow chart of an example method of capturing images including research participants wearing wearable cameras.

FIG. 14 is a flow chart of an example method of capturing screen shots of displays viewed by research participants.

FIG. 15 is a flow chart of an example method of capturing images including a police officer wearing a wearable camera.

FIG. 16 is a flow chart of an example method of capturing images from a dashboard camera of a police officer.

Corresponding reference numerals indicate corresponding features throughout the several views of the drawings.

### DETAILED DESCRIPTION

Example embodiments will now be described more fully with reference to the accompanying drawings.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

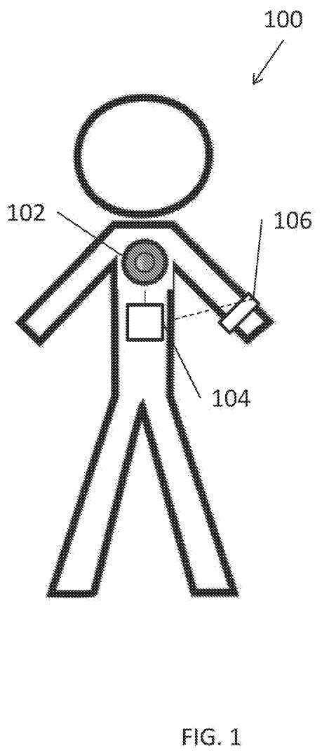

A system for capturing images based on a user's biorhythms according to one example embodiment of the present disclosure is illustrated in FIG. 1 and indicated generally by reference number 100. As shown in FIG. 1, the system 100 includes a camera 102, a processor 104 and a biosensor 106. The biosensor 106 detects (e.g., senses) a biorhythm of the user.

The processor 104 is configured to receive a biorhythm signal from the biosensor 106, where the biorhythm signal is indicative of the biorhythm as detected by the biosensor 106. The processor 104 compares the received biorhythm signal to a biorhythm threshold and, in response to the received biorhythm signal exceeding the biorhythm threshold, triggers the camera 102 to capture one or more images. As described herein, “images” may refer to any number of pictures, videos, etc.

Accordingly, the system 100 allows for a user to capture (e.g., obtain, record, take, snap, etc.) one or more images (e.g. pictures, videos, etc.) at times corresponding to biorhythm changes of the user. The camera 102 can be triggered to capture images when the user experiences an emotional, physical, etc. response as indicated by measured biorhythm(s) of the user. Significant moments can be captured automatically without requiring the user to actively control the camera 102 to capture images.

The user can thereby proceed with an activity, go throughout the day, etc. while the camera 102 automatically captures moments where the user is happy, sad, surprised, startled, excited, experiencing physical exertion, etc. Because these types of moments are more likely to correspond to interesting experiences for the user, triggering the camera 102 to capture pictures and/or videos when the user's biorhythm(s) change can provide a record of significant experiences with little to no interaction required between the user and the camera. The system 100 may allow for capturing of significant moments, experiences, etc. that may otherwise be missed by the user.

The system 100 can be used in any suitable setting as further described below. For example, the system 100 may include one or more wearable cameras 102, one or more remote cameras 102 (e.g., positioned away from the user's body), etc. that may be networked together.

Any suitable biosensor(s) 106 may be incorporated to detect one or more biorhythm(s) of the user. One or more processors 104 can be configured to trigger camera(s) 102 to capture pictures and/or videos based on multiple different biorhythm thresholds. In some embodiments, captured images can be transferred to servers, monitoring devices, etc., so that users can store the captured images, third parties can review the captured videos, etc.

Example applications will be described further below and include, but are not limited to, elderly persons and caretakers, parents of young children, teachers and children in schools, amusement park guests, pets and owners, research focus groups, police officers, etc.

Referring again to FIG. 1, the biosensor 106 (e.g., biorhythm sensor) is illustrated as part of a wristband. However, any suitable biosensor(s) 106 can be used in the system 100. For example, the biosensor 106 could include a watch biosensor, a bracelet biosensor, clip-on earring biosensor(s), earbud biosensor(s), a band-aid biosensor, etc.

In some embodiments, the biosensor 106 may be built into part of a user's glasses (e.g., smart glasses), such as in the arm(s) of the glasses. In those cases, the user's heart rate may be captured by the biosensor 106 from behind the user's ear. In this form factor, the camera 102 could be built into a frame of the glasses (e.g., above the lenses, near the lenses, etc.). Captured images could be transmitted via WiFi to a server, etc.

The biosensor(s) 106 can be configured to detect any suitable biorhythm of the user. For example, the biosensor 106 may include an optical heart rate sensor configured to capture beats per minute of the user (i.e., heart rate), heart rate variability, etc. The biosensor 106 may include an electrodermal activity (EDA) sensor configured to measure skin conductance, a blood pressure sensor, etc. In some embodiments, the biosensor 106 may be configured to detect respiration rate.

The processor 104 is configured to trigger the camera 102 to capture images in response to the received biorhythm signal exceeding a biorhythm threshold. If the detected biorhythm is a heart rate, the processor 104 may be configured to trigger the camera 102 to capture images when the detected heart rate signal exceeds a threshold. In some embodiments, the threshold could be set relative to the resting heart rate of the user. For example, if the user's resting heart rate is sixty beats per minute, the threshold may be set to ninety beats per minute.

The biorhythm threshold may be set using any suitable programming of the processor 104, a potentiometer, etc. In some embodiments, an algorithm could detect one or more personal metrics of a user (e.g., by detecting a user's individual resting heat rate over a period of time), and then establish a biorhythm threshold unique to the user based on the detected information.

As used herein, a biorhythm signal “exceeding” a biorhythm threshold may refer to the biorhythm signal increasing above the biorhythm threshold when the biorhythm signal is an upper limit (e.g., indicative of an excited user), may refer to the biorhythm signal decreasing below the biorhythm threshold when the biorhythm signal is below a lower limit (e.g., indicative of a potential health issue), may refer to the biorhythm signal moving outside a range bounded by an upper and a lower biorhythm signal, etc.

For example, the camera 102 can be triggered to capture images when a heart rate is detected below a biorhythm threshold, such as an unusually low heart rate, no detected heart rate, etc. This may be useful in applications including elderly patients, police officers, etc.

As mentioned above, heart rate variability may be used for the biorhythm signal. The heart rate variability can be based on measurement of variation in a time interval between heart beats of the user. The biorhythm threshold(s) can be set based on changes in this interval (e.g., indicating variability in the user's heart rate) such that the camera 102 is triggered to capture images when the user's heart rate varies beyond the threshold.

In some embodiments heart rate variability can be configured to indicate multiple (e.g., two, three, four, etc.) distinct emotions. The processor 104 can be configured to trigger the camera 102 to capture images only when the user is within a subset of range(s) of the distinct emotions. For example, the processor 104 could be configured to capture images only when the user is within a range of one of the distinct emotions (e.g., happy), within ranges of two of the distinct emotions (e.g. happy and surprised). In some embodiments, the processor may be configured to analyze heart rate variability by detecting low frequency heart rate signal, high frequency heart rate signals, comparing different frequency heart rate signals to one another, etc.

The biosensor 106 can transmit biorhythm signals to the processor 104 using any suitable communication medium, including a wireless communication medium (e.g., BLUETOOTH, WiFi, cellular, ANT+, etc.) and/or a wired connection. Although FIG. 1 illustrates the processor 104 as coupled to but separate from the camera 102, in other embodiments the processor 104 may be part of the camera 102 (e.g., in a smartphone camera), may be separate from the camera 102, may be housed in the biosensor 106, etc. For example, the biosensor 106 may include the processor 104, may be coupled to the processor 104, etc., and the processor 104 may send a trigger signal to the camera 102 via a wireless communication medium and/or a wired connection.

In FIG. 1, the illustrated camera 102 is a wearable camera. Any suitable wearable camera or combination of wearable cameras can be used in the system 100. For example, the camera 102 may include one or more cameras incorporated into a computing device (e.g. a smartphone camera), a standalone camera, etc. In some embodiments, the camera 102 may include a clip, magnet, etc. to allow the camera 102 to be worn by the user in various ways. For example, the user may wear the camera 102 on the torso in an outward facing manner. Other examples include, but are not limited to, camera(s) clipped to a hat of the user (e.g., bill of a baseball cap), camera(s) attached to the helmet of a user, camera(s) attached to a shirt sleeve of a user, camera(s) attached to glasses of the user (e.g., a doctor's glasses in the operating room), camera(s) formed as part of a contact lens, camera(s) coupled to a wristband, etc.

In some embodiments, at least two cameras 102 may be coupled to a wristband (e.g., integrated into the wristband, etc.). The processor 104 may be configured to trigger the at least two cameras 102 to capture one or more images (e.g., pictures and/or videos) when the biorhythm signal exceeds the biorhythm threshold. The processor may be configured to combine the one or more images captured by the at least two cameras using any suitable image/video combination techniques (e.g., stitching the images together, etc.).

Some of the examples listed above include examples of outward facing cameras 102, which could capture images from the view point of the user when triggered. For example, such outward facing cameras could capture images of what the user is looking at when triggered based on changes in the user's biorhythm. As should be apparent, other wearable cameras may be adapted to face toward the user such that the camera(s) could capture an image of the user's face, body, etc. when triggered based on changes in the user's biorhythm. Wearable cameras 102 facing toward the user could be helpful in capturing a user's facial expressions, reactions, etc. during significant moments.

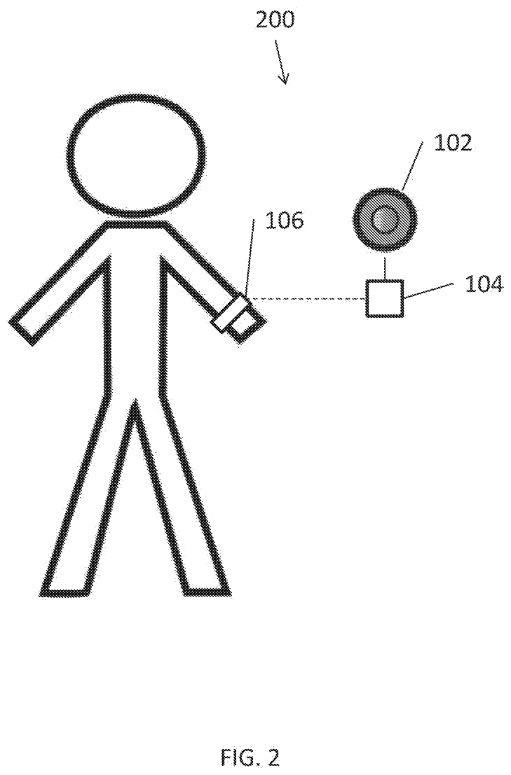

FIG. 2 illustrates a system 200 for capturing images based on a user's biorhythms according to another example embodiment of the present disclosure. The system 200 is similar to the system 100 of FIG. 1, but the camera 102 is remote from the user. Accordingly, the processor 104 can be configured to trigger a remote camera to capture images when a received biorhythm signal from the biosensor 106 exceeds a biorhythm threshold.

The camera 102 can be any suitable camera capable of being positioned remote from the user. In some embodiments the camera 102 is a stationary camera. For example, the camera may be a surveillance camera, etc. mounted on a wall, a tripod, any other suitable fixture, etc. Alternatively, the camera 102 could be affixed to a moveable object remote from the user, such as a drone, etc.

The camera 102 may be oriented towards the user in order to capture images of the user when triggered based on the biorhythm signal. For example, a home, classroom, etc. camera 102 may be positioned to capture images of family members, students, pets, etc. when triggered. Surveillance cameras in an amusement park or other setting may be triggered to capture images of the user based on the biorhythm signal. Sports teams may use cameras to assist in player evaluation by capturing images of players based on biorhythm signals. Some embodiments may include cameras 102 having wide-angle lenses to increase the capture range of the camera. Some cameras 102 may rotate to track the user based on other identifications of the user, such as an identification tag. For example, a user could wear a tag causing a rotatable camera to move and track the user to keep the user in the frame of the camera 102.

In some embodiments, multiple remote and/or wearable cameras may be used together in a network. For example, a home, classroom, school, amusement park, etc. may include multiple stationary cameras 102 located at different positions within a space. The biosensor 106, processor 104, other identifiers, etc. may communicate with the network of cameras 102 to trigger a closest camera to the user to capture images based on the biorhythm signal.

For example, when an amusement park guest biorhythm signal exceeds a threshold, the biosensor 106, processor 104, etc. may trigger the nearest camera 102 to the user to capture an image. Accordingly, the guest may proceed throughout the park during the day and the appropriate camera 102 will be triggered to capture an image of the user when the user's biorhythm exceeds a threshold. Similarly, a network of cameras 102 could be disposed in a school, home, etc., and the processor 104 could trigger the appropriate camera 102 (e.g. a camera in the same room as the user) to take a picture when the user's biorhythm exceeds a threshold.

Further, wearable cameras and remote cameras can be used together in the same system to capture both a users viewpoint and a remote image of the user at the same time when the user's biorhythm exceeds a threshold. For example, in a school setting a student's wearable camera could capture images at the same time a classroom camera is capturing images to provide multiple views of a significant event for the student. As another example, a police officer could have a wearable camera and a dashboard mounted camera that both capture images when the police officer's biorhythm exceeds a threshold.

In some embodiments, a network of cameras could be linked so that when one camera is triggered to capture images, all other cameras in the network are also triggered to capture images. For example, in a police setting, if one officer's camera is triggered to capture images, police cameras of other officers could also be triggered to capture images. These networks may be pre-established. The cameras in the network could be linked through a near-field communication protocol (e.g., BLUETOOTH, ANT+), could be linked through geographic location (e.g., all cameras within a defined location based on GPS signals), etc. As should be apparent, any suitable combination of wearable and/or remote cameras may be used without departing from the scope of the present disclosure.

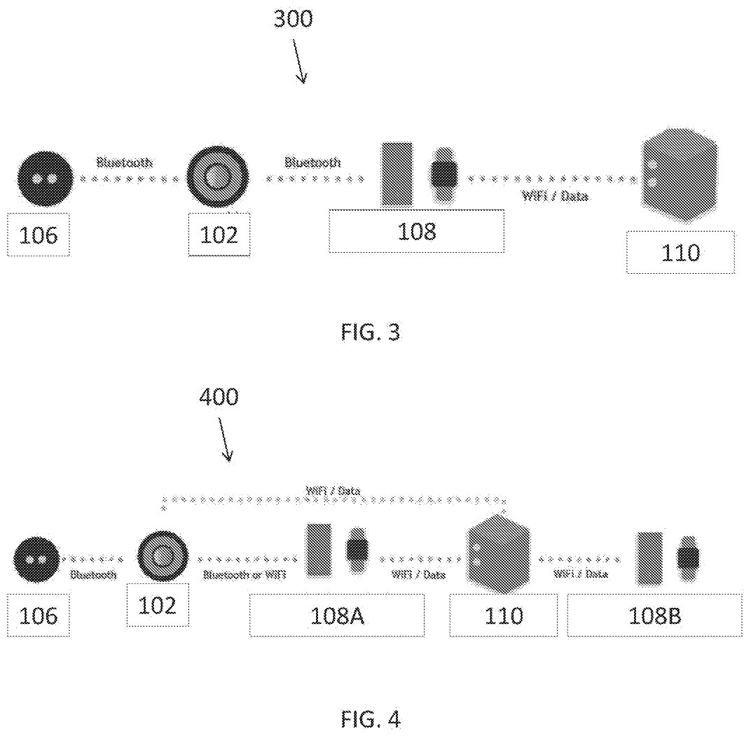

Referring now to FIG. 3, an example system 300 includes a biosensor 106 in communication with a camera 102. The camera 102 is in communication with a monitoring device 108, which is in communication with a server 110. Although FIG. 3 illustrates BLUETOOTH and WiFi/Data communications, any suitable communication protocols may be used between components of the system 300.

The system 300 is similar to the systems 100 and 200 of FIGS. 1 and 2, but further includes the monitoring device 108 and the server 110. The camera is configured to transmit captured images to the monitoring device 108 after they are taken based on a user's biorhythm signal exceeding a threshold.

The monitoring device 108 allows a third party (e.g., authorized user, monitor, etc.) to view images taken by the camera 102, thereby allowing the third party to monitor the user. For example, an elderly parent may wear a biosensor 106 and wearable camera 102 as described above in the system 100. When image(s) are captured based on the elderly parent's biorhythm exceeding a threshold, the image(s) may be transmitted to a monitoring device 108 of a child of the elderly parent so that the child can monitor the situation to make sure the elderly parent is safe. Similarly, young children can wear a biosensor 106 and wearable camera 102, and captured images can be transmitted to a parent's monitoring device 108.

As another example, video from a police camera could be streamed to a remote location such as a police dispatch, a police headquarters, a district office, etc. This would allow the streamed video to be monitored by third parties at the remote locations. Additionally, or alternatively, the video from the police camera could be transmitted to other officer devices such as a patrol car computer, an officer tablet computer, etc.

The monitoring devices 108 can include suitable computing devices for receiving and displaying captured images. For example, smartphones, smartwatches, tablets, computers, etc. may be used as monitoring devices 108. Monitoring devices 108 can be used by third parties to monitor activity of the user wearing the biosensor 106. Alternatively, or in addition, the user wearing the biosensor 106 may have a monitoring device to review their own captured images, to store captured images, to facilitate transfer of captured images to a server 110, etc.

FIG. 4 illustrates an example system 400 similar to the system 300 of FIG. 3, but further including an additional monitoring device 108B connected to the server 110. This allows for multiple third parties to monitor captured images of the user based on the user's biorhythm exceeding a threshold.

For example, an elderly person's captured images may be transmitted to both a child's monitoring device 108A and a server 110. A caretaker of the elderly person may also receive the captured images via server 110 on the caretaker's monitoring device 1088. This can provide additional levels of monitoring and review for multiple third parties.

Accordingly, the camera 102 may transmit captured images directly to a monitoring device 108 and/or directly to a server 110 (e.g., cloud server, etc.). As another example, an amusement park guest may have captured images transferred to their own monitoring device 108A, as well as a server 110 that can be accessed by another third party's monitoring device 1088 (e.g., parents of amusement park guest children, etc.).

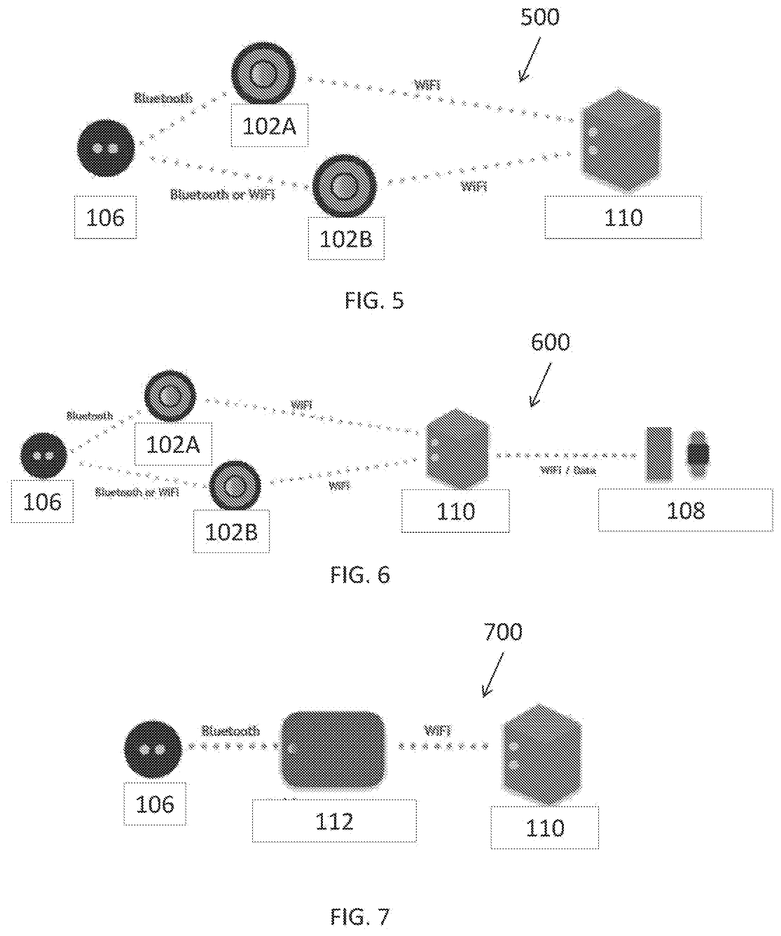

FIG. 5 illustrates an example system 500 including a biosensor 106 coupled to a wearable camera 102A and a remote camera 1028. Both cameras 102A and 102B transmit captured images to a server 110. This allows for capturing of images from both a wearable camera 102A and a remote camera 102B to obtain multiple images when a user's biorhythms exceed a threshold.

As an example, teachers and students may wear individual cameras 102A, while also in view of a remote classroom camera 102B positioned in the room. The wearable cameras 102A and remote classroom camera 1028 can each capture images of the teachers and students and upload the pictures and/or videos to a server 110 for the school's use.

Similarly, a pet in the home may wear a camera 102A, and also have images captured from a remote room camera 1028. These images can be uploaded to a server 110 for an owner of the pet to view. As another example, a police officer can have a wearable camera 102A and a dashboard camera 1028 that both transmit captured images to a police force server 110.

FIG. 6 illustrates an example system 600 similar to the system 500, but further including a monitoring device 108 in communication with the server 110. This allows a third party to review images captured from the cameras 102A and 102B to monitor a user wearing the biosensor 106. With regard to the examples described above relative to FIG. 5, the monitoring device can be used by parents to view captured images of their student children, be used by owners to view captured images of their pets, etc.

FIG. 7 illustrates an example system 700 including a biosensor 106, a display device 112 and a server 110. The display device 112 is configured to display images to a user and to capture screen shots of the display device 112 when the user's biorhythm exceeds a threshold. Accordingly, the display device 112 can capture screen images that are determined to be significant based on a user's biorhythm response when viewing the screen images. The display device 112 may be any suitable device capable of displaying images to the user and capturing screen shots of displayed images (e.g., screen images), including but not limited to tablets, computers, smartphones, smart televisions, etc.

As an example, the system 700 can be used by market research focus groups, testing agencies, etc. to display images to participants in research (e.g., field research, etc.). When the participants view a display image that causes a significant reaction as detected by a biorhythm change, the display device 112 can capture a screen shot. This allows the research group (e.g., advertisers, etc.) to determine which display images create the strongest reactions. Alternatively, or in addition, a research group may use any one or more of example systems 300-600 described above to capture images of participants who are part of a study when the participant's biorhythm exceeds a threshold.

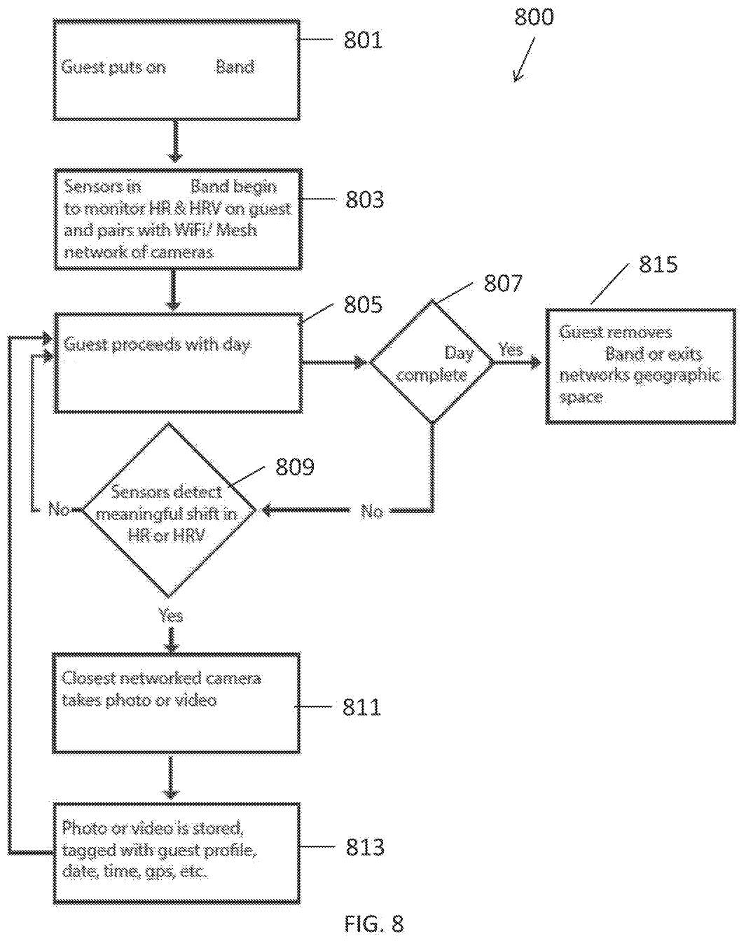

FIG. 8 illustrates an example method 800 according to another example embodiment of the present disclosure. At 801, an amusement park guest puts on a wristband biosensor. At 803, sensors in the band start to monitor a heart rate (HR) and heart rate variability (HRV) of the guest and pair with a WiFi/Mesh network of cameras.

As the guest proceeds with the day at 805 and until the day is complete at 807, the biosensor continually monitors the HR and/or HRV of the guest to detect meaningful shifts in the HR and/or HRV at 809. If a meaningful shift is detected (e.g., the biorhythm signal exceeds a threshold), the closest network camera takes photo or video at 811. The phone or video is stored, tagged with a guest profile, date, time, GPS location, etc., at 813.

Once the day is over at 815, the guest can remove the wristband biosensor and exit the network geographic space of the amusement park. Accordingly, the method 800 allows guests to obtain captured images from cameras positioned through the park during their visit.

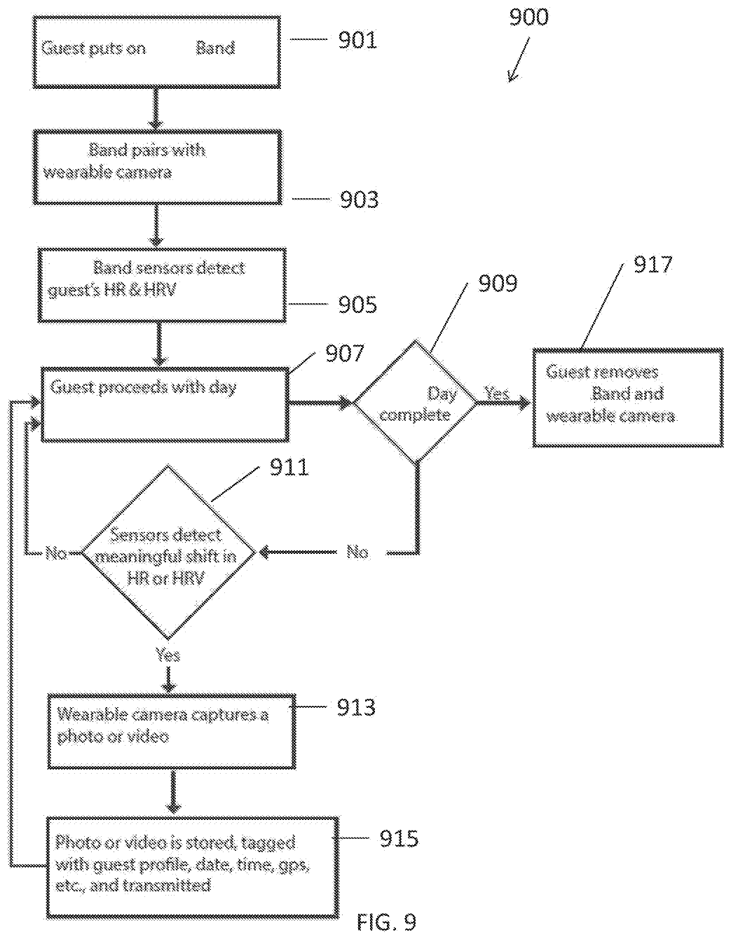

FIG. 9 illustrates a method 900 that is similar to the method 800 of FIG. 8, but uses a wearable camera for the guest. At 901, an amusement park guest puts on a wristband biosensor. At 903, the wristband pairs with a wearable camera worn by the guest. At 905, sensors in the band start to monitor a heart rate (HR) and heart rate variability (HRV) of the guest.

As the guest proceeds with the day at 907 and until the day is complete at 909, the biosensor continually monitors the HR and/or HRV of the guest to detect meaningful shifts in the HR and/or HRV at 911. If a meaningful shift is detected (e.g., the biorhythm signal exceeds a threshold), the wearable camera takes photo or video at 913. The photo or video is stored, tagged with a guest profile, date, time, GPS location, etc. at 915.

Once the day is over at 917, the guest can remove the wristband biosensor and wearable camera. Accordingly, the method 900 allows guests to obtain captured images from a wearable camera during their visit.

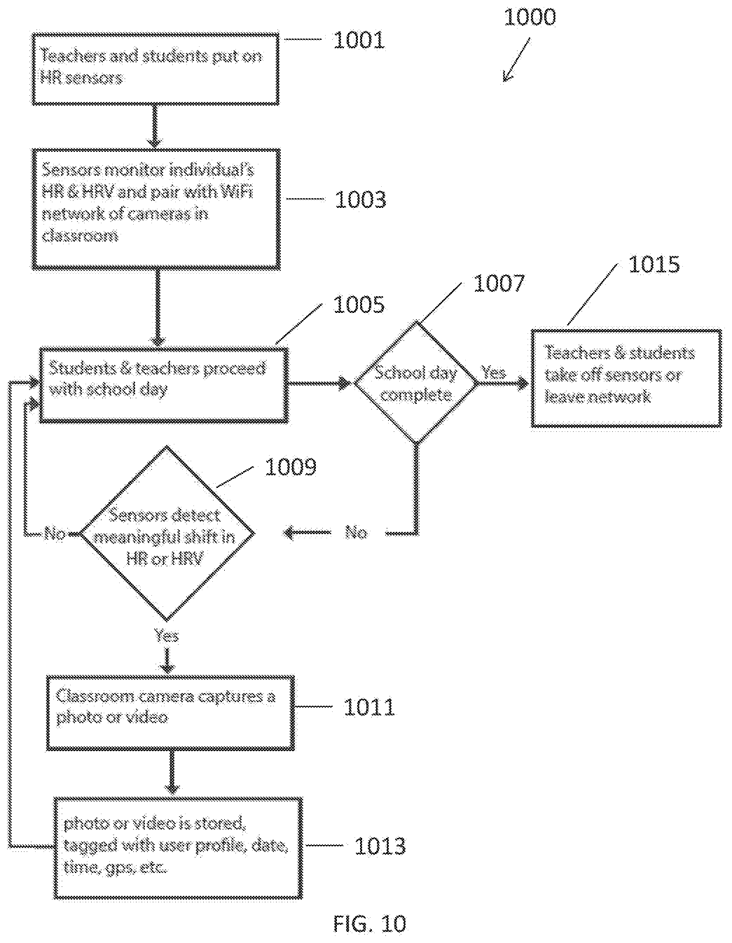

FIG. 10 illustrates an example method 1000 according to another example embodiment of the present disclosure. At 1001, teachers and students put on HR sensors. At 1003, the sensors monitor an individual's heart rate (HR) and heart rate variability (HRV) and pair with a WiFi network of cameras in the classroom.

As the students and teachers proceed with the school day at 1005 and until the day is complete at 1007, the biosensor continually monitors the HR and/or HRV to detect meaningful shifts in the HR and/or HRV at 1009. If a meaningful shift is detected (e.g., the biorhythm signal exceeds a threshold), a classroom camera captures photo or video at 1011. The image or video is stored, tagged with a user profile, date, time, GPS location, etc. at 1013.

Once the day is over at 1015, the teachers and students can remove the biosensors and leave the network. Accordingly, the method 1000 allows teachers and students to obtain captured images from cameras positioned in the classroom during the school day.

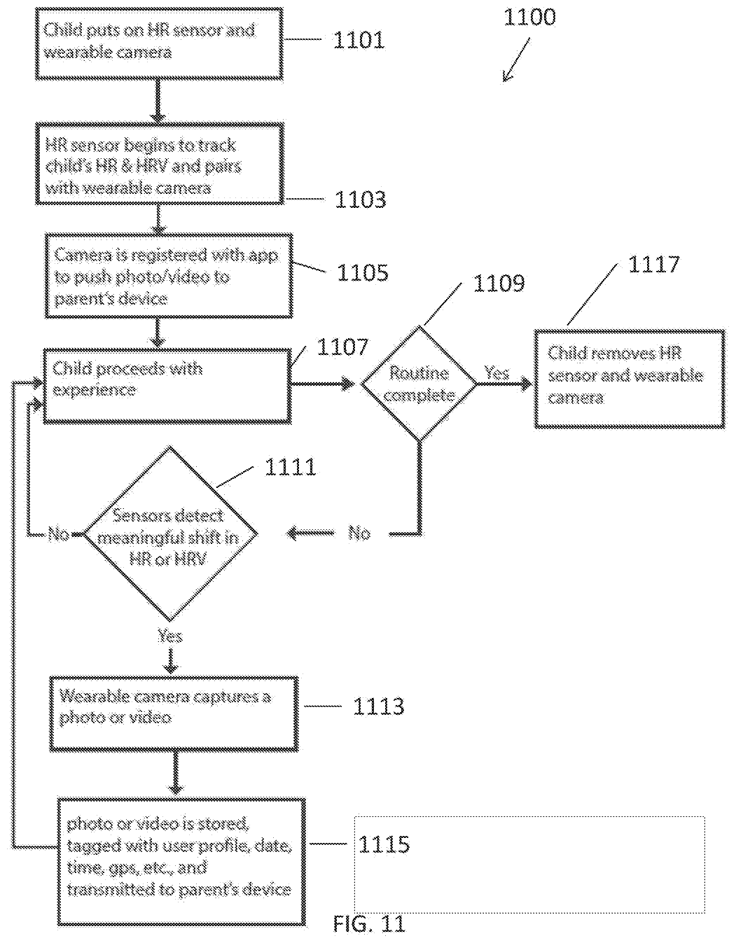

FIG. 11 illustrates a method 1100 according to another example embodiment of the present disclosure. At 1101, a child puts on a HR sensor and wearable camera. At 1103, the HR sensor starts to monitor a heart rate (HR) and heart rate variability (HRV) of the child and pairs with the wearable camera. The camera is registered with an application to push captured photos and videos to a parent's device at 1105.

As the child proceeds with an experience (e.g., activity, period of time, etc.) at 1107 and until the experience is complete at 1109, the biosensor continually monitors the HR and/or HRV of the child to detect meaningful shifts in the HR and/or HRV at 1111. If a meaningful shift is detected (e.g., the biorhythm signal exceeds a threshold), the wearable camera takes photo or video at 1113. The photo or video is stored, tagged with a user profile, date, time, GPS location, etc. at 1115 and transmitted to a parent's monitoring device.

Once the experience is over at 1117, the child can remove the wristband biosensor and wearable camera. Accordingly, the method 1100 allows parents to obtain captured images from their child's from wearable camera. Similarly, the method 1100 can be used for caretakers of elderly persons to monitor the elderly persons.

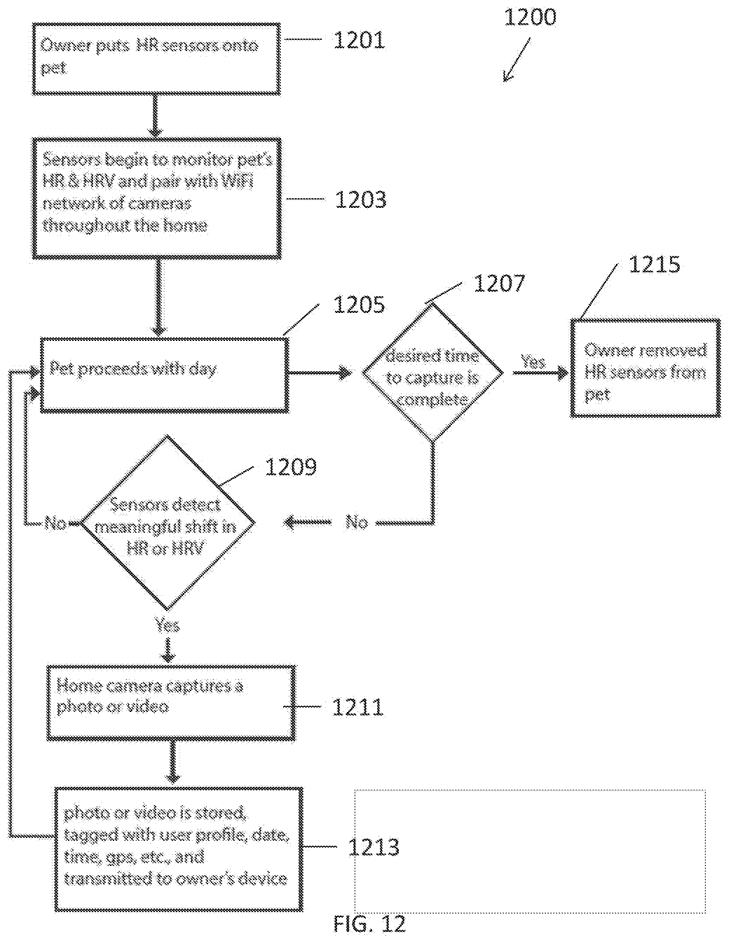

FIG. 12 illustrates an example method 1200 according to another example embodiment of the present disclosure. At 1201, an owner puts HR sensor onto a pet. At 1203, the sensors monitor the pet's heart rate (HR) and heart rate variability (HRV) and pair with a WiFi network of cameras in the home.

As the pet proceeds with the day at 1205 and until the day is complete at 1207, the biosensor continually monitors the HR and/or HRV to detect meaningful shifts in the HR and/or HRV at 1209. If a meaningful shift is detected (e.g., the biorhythm signal exceeds a threshold), a home camera captures photo or video at 1211. The photo or video is stored, tagged with a user profile, date, time, GPS location, etc., at 1213, and is transmitted to the owner's device.

Once the day is over at 1215, the owner removes the biosensor from the pet. Accordingly, the method 1200 allows owners to obtain captured images of pets. A similar method may also be used for caretakers of elderly person's in a medical facility, market research with stationary cameras in a focus group facility, mock trial courtrooms, etc. Although the method 1200 describes removal of the biosensor from the pet at 1215, in some embodiments the biosensor may be left on the pet for more than one day. For example, the biosensor could be built into a collar of the pet, which would not typically be put on and taken off on a daily basis.

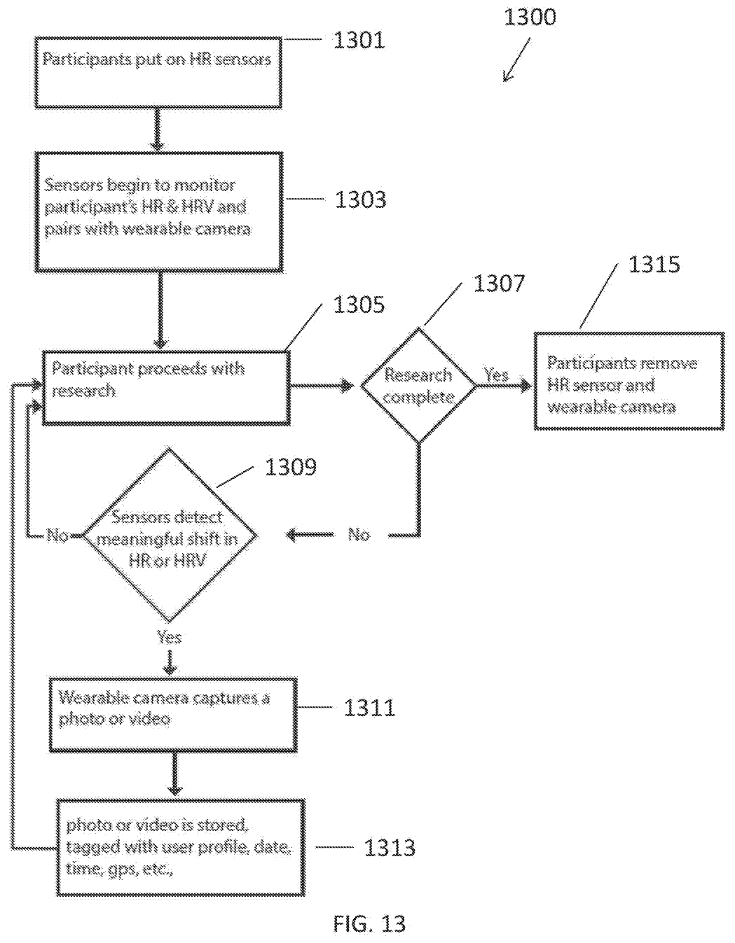

FIG. 13 illustrates an example method 1300 according to another example embodiment of the present disclosure. At 1301, research participants put on HR sensors. At 1303, the sensors monitor the participant's heart rate (HR) and heart rate variability (HRV) and pair with a wearable camera.

As the participant proceeds with the research at 1305 and until the research is complete at 1307, the biosensor continually monitors the HR and/or HRV to detect meaningful shifts in the HR and/or HRV at 1309. If a meaningful shift is detected (e.g., the biorhythm signal exceeds a threshold), the wearable camera captures photo or video at 1311. The photo or video is stored, tagged with a user profile, date, time, GPS location, etc. at 1313.

Once the research is complete at 1315, the participant removes the HR sensor and wearable camera. Accordingly, the method 1300 allows a market research group to obtain images from wearable cameras of research participants during a study.

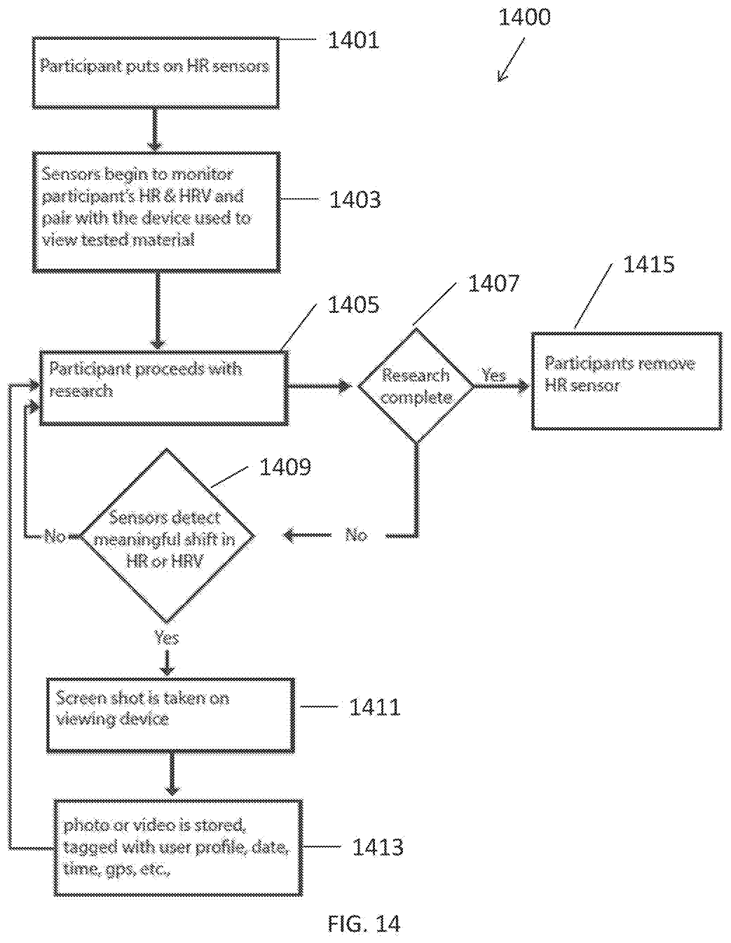

FIG. 14 illustrates an example method 1400 according to another example embodiment of the present disclosure. At 1401, research participants put on HR sensors. At 1403, the sensors monitor the participant's heart rate (HR) and heart rate variability (HRV) and pair with a display device used to display tested material on a screen of the display device.

As the participant proceeds with the research at 1405 and until the research is complete at 1407, the biosensor continually monitors the HR and/or HRV to detect meaningful shifts in the HR and/or HRV at 1409. If a meaningful shift is detected (e.g., the biorhythm signal exceeds a threshold), a screen shot is taken on the viewing device at 1411 (e.g., a current screen image of the display device is captured). The screen shot is stored, tagged with a user profile, date, time, GPS location, etc. at 1413.

Once the research is complete at 1415, the participant removes the HR sensor. Accordingly, the method 1400 allows a market research group to obtain screen shots of display device images viewed by research participants during a study.

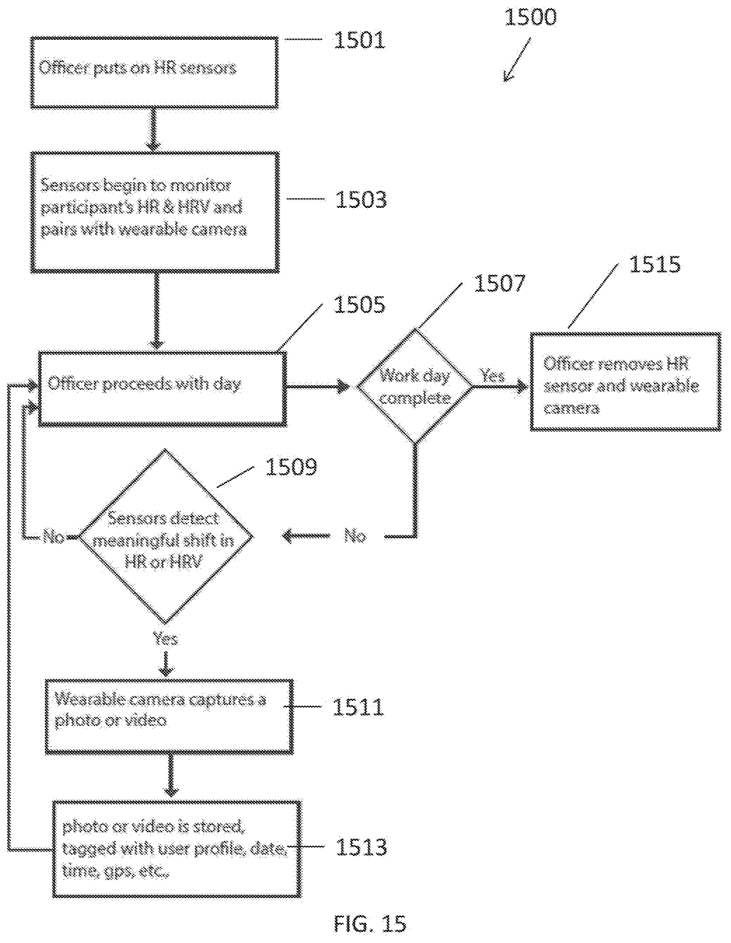

FIG. 15 illustrates an example method 1500 according to another example embodiment of the present disclosure. At 1501, police officers put on HR sensors. At 1503, the sensors monitor the participant's heart rate (HR) and heart rate variability (HRV) and pair with a wearable camera.

As the officer proceeds with the day at 1505 and until the work day is complete at 1507, the biosensor continually monitors the HR and/or HRV to detect meaningful shifts in the HR and/or HRV at 1509. If a meaningful shift is detected (e.g., the biorhythm signal exceeds a threshold), the wearable camera captures photo or video at 1511. The photo or video is stored, tagged with a user profile, date, time, GPS location, etc. at 1513.

Once the work day is complete at 1515, the police officer removes the HR sensor and wearable camera. Accordingly, the method 1500 allows for capturing of images from wearable cameras of police officers.

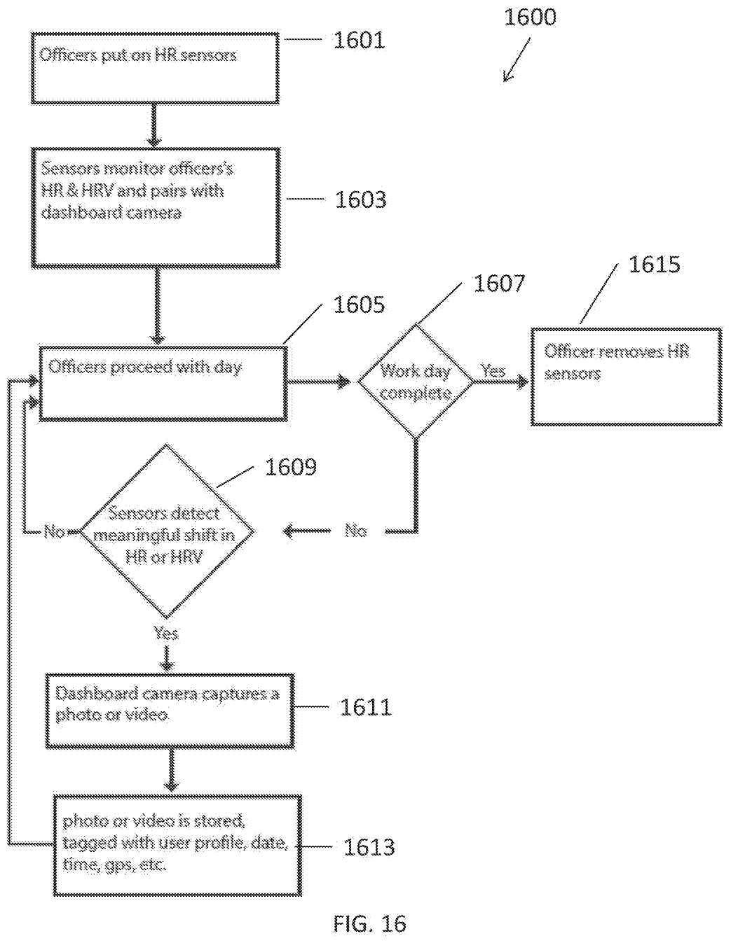

FIG. 16 illustrates an example method 1600 according to another example embodiment of the present disclosure. At 1601, police officers put on HR sensors. At 1603, the sensors monitor the participant's heart rate (HR) and heart rate variability (HRV) and pair with a dashboard camera.

As the officer proceeds with the day at 1605 and until the work day is complete at 1607, the biosensor continually monitors the HR and/or HRV to detect meaningful shifts in the HR and/or HRV at 1609. If a meaningful shift is detected (e.g., the biorhythm signal exceeds a threshold), the dashboard camera captures photo or video at 1611. The photo or video is stored, tagged with a user profile, date, time, GPS location, etc. at 1613.

Once the work day is complete at 1615, the police officer removes the HR sensor. Accordingly, the method 1500 allows for capturing of images from dashboard cameras of police officers.

According to another example embodiment of the present disclosure, a method for capturing images based on biorhythms is disclosed. The method generally includes sensing a biorhythm of a user with a biosensor, and transmitting a biorhythm signal from the biosensor to at least one processor. The biorhythm signal is indicative of at least one biorhythm of a user as detected by the biosensor. The method also includes comparing the biorhythm signal to a biorhythm threshold, and, in response to the biorhythm signal exceeding the biorhythm threshold, the at least one processor performing at least one of: triggering a camera disposed remotely from the biosensor and the user to capture one or more images; triggering a wearable camera to capture one or more images and transmitting the one or more images captured by the wearable camera to the monitoring device; and capturing a current screen image of a computing device display.

The cameras, processors, monitoring devices, servers, display devices, etc. described herein may be configured to perform the described actions using any suitable hardware and/or software implementations. For example, appropriate circuitry, logic, etc. may implemented in hardware to perform methods described herein. Similarly, appropriate computer-executable instructions may be stored in memory, executed by a processor, etc. to perform the methods described herein. As should be apparent, any methods described herein can be implemented on any suitable systems, and any systems described herein may implement any suitable methods. Further, components, systems, methods, etc. described herein can be combined in any suitable manner without departing from the scope of the present disclosure.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

1. A system for capturing images based on biorhythms, the system comprising:
at least one processor configured to receive a biorhythm signal from a biosensor, the received biorhythm signal indicative of at least one biorhythm of a user as detected by the biosensor; and
a camera disposed remotely from the biosensor and user and adapted to capture one or more images when triggered by the at least one processor, the at least one processor configured to compare the received biorhythm signal to a biorhythm threshold and, in response to the received biorhythm signal exceeding the biorhythm threshold, to trigger the camera to capture the one or more images.

2. The system of claim 1, wherein the camera is a stationary camera.

3. The system of claim 1, further comprising multiple cameras including said camera, the multiple cameras disposed remotely from the biosensor and the user.

4. The system of claim 3, wherein the at least one processor is configured to trigger the closest one of the multiple cameras to the user to capture the one or more images.

5. The system of claim 1, wherein the triggered camera is oriented towards the user such that the camera captures the user in the one or more images when triggered by the at least one processor.

6. The system of claim 1, wherein the biorhythm signal includes a heart rate of the user.

7. The system of claim 1, wherein the biorhythm threshold includes at least one of a heart rate value and a heart rate variability.

8. The system of claim 1, wherein the biosensor includes at least one of a heart rate sensor, an electrodermal activity sensor, and a blood pressure sensor.

9. The system of claim 1, wherein the biosensor is housed in at least one of a wristband, a watch, a bracelet, an earring, an earbud, glasses and a band-aid.

10. The system of claim 1, wherein the at least one processor is configured to receive the biorhythm signal from the biosensor via a wireless connection.

11. A system for capturing images based on biorhythms, the system comprising:
at least one processor configured to receive a biorhythm signal from a biosensor, the received biorhythm signal indicative of at least one biorhythm of a user as detected by the biosensor;
a camera adapted to capture one or more images when triggered by at least one processor; and
a monitoring device configured to receive the one or more captured images, the at least one processor configured to compare the received biorhythm signal to a biorhythm threshold, to trigger the camera to capture the one or more images in response to the received biorhythm signal exceeding the biorhythm threshold, and to transmit the one or more captured images to the monitoring device.

12. The system of claim 11, wherein the camera comprises a wearable camera adapted to be worn by the user.

13. The system of claim 12, wherein the wearable camera is adapted to be worn on at least one of a torso of the user, a hat of the user, a wristband of the user, a helmet of the user, a shirt sleeve of the user, glasses of the user, and a contact lens of the user.

14. The system of claim 12, wherein the wearable camera includes two cameras coupled to a wristband.

15. The system of claim 12, wherein the wearable camera includes at least two cameras, the at least one processor is configured to trigger the at least two cameras to capture the one or more images when the biorhythm signal exceeds the biorhythm threshold, and the at least one processor is configured to combine the one or more images captured by the at least two cameras.

16. The system of claim 11, wherein the camera comprises a stationary camera disposed remotely from the biosensor and the user.

17. The system of claim 11, wherein the biorhythm signal includes a heart rate of the user, and the biorhythm threshold includes at least one of a heart rate value and a heart rate variability.

18. The system of claim 11, wherein:
the biosensor includes at least one of a heart rate sensor, an electrodermal activity sensor, and a blood pressure sensor; and
the biosensor is housed in at least one of a wristband, a watch, a bracelet, an earring, an earbud, glasses and a band-aid.

19. The system of claim 11, wherein the at least one processor is configured to receive the biorhythm signal from the biosensor via a wireless connection.

20. A system for capturing images based on biorhythms, the system comprising:
at least one processor configured to receive a biorhythm signal from a biosensor, the received biorhythm signal indicative of at least one biorhythm of a user as detected by the biosensor; and
a computing device display adapted to display multiple screen images, the at least one processor configured to compare the received biorhythm signal to a biorhythm threshold and, in response to the received biorhythm signal exceeding the biorhythm threshold, to capture a current screen image of the computing device display.

21-25. (canceled)
